(19) United States
(12) Patent Application Publication
(10) Pub. No.: US 2017/0266237 A1
(43) Pub. Date: Sep. 21, 2017

(54) Generation of Brown Adipose Tissue (BAT) from Mesenchymal Cells

(71) Applicants: National University of Singapore, Singapore (SG); Auckland Uniservices LTD, Auckland (NZ)

(72) Inventors: Michael Raghunath, Singapore (SG); Michelle Hui Ching Lee, Singapore (SG); Allan Sheppard, Auckland (NZ)

(21) Appl. No.: 15/443,842

(22) Filed: Feb. 27, 2017

Related U.S. Application Data
(63) Continuation of application No. 14/384,565, filed on Sep. 11, 2014.
(60) Provisional application No. 61/609,456, filed on Mar. 12, 2012.

(51) Int. Cl. A61K 35/35; A61K 45/06; C12N 5/077

(52) U.S. Cl. CPC A61K 35/35; C12N 5/0653; C12N 2500/38; C12N 2501/155; C12N 2501/33; C12N 2501/385; C12N 2501/39; C12N 2533/70; C12N 2501/01; A61K 45/06; C12N 2501/00; C12N 2500/34; C12N 2506/1353

(57) ABSTRACT

Methods of generating functional human brown adipocytes, comprising exposing human stem cells, progenitor cells, or white adipocytes to culture with an differentiation cocktail that comprises one or more browning agents (e.g., one or more macromolecular crowders), and optionally one or more adipogenic agents, are described, as are populations of human brown adipocytes generated by the methods, and uses for the populations. Methods of generating functional human brown adipocytes in an individual, such as by administering a pharmaceutical composition comprising an differentiation cocktail, are also described.

### RELATED APPLICATION

This application claims the benefit of U.S. Provisional Application No. 61/609,456, filed on Mar. 12, 2012. The entire teachings of the above application are incorporated herein by reference.

### BACKGROUND OF THE INVENTION

Over 1 billion adults are either overweight or obese BMI and more than 150 million adults have diabetes, most of which is type 2 diabetes driven by obesity-associated insulin resistance, reviewed in Cypess, A. M. and Kahn, C. R., Curr. Opin. Endocrin. Diabetes & Obesity 17, 143-149 (2010). 25% of children in the USA are also now overweight or obese leading to the appearance of type 2 diabetes in this previously unaffected population. These numbers are expected to increase by more than half again by the year 2025 worldwide, with especially severe impact in less developed countries. The Health Promotion Board (HPB) in Singapore revealed that obesity in Singapore has increased to 10.8 per cent, up from 6.9 per cent in 2004. In 23 of 45 Asia Pacific countries, diabetes will affect >10% of the population by 2025; China: 24 million >20 in 2007—47 million forecasted 2025 India: 14% 36 million to 73 million Singapore: 30%; (International Diabetes Federation 2009).

### SUMMARY OF THE INVENTION

The present invention pertains to methods of generating functional human brown adipocytes from human stem cells, progenitor cells, or white adipocytes, by culturing cells with a differentiation cocktail. When stem cells or progenitor cells are used, the differentiation cocktail comprises one or more browning agents (e.g., macromolecular crowder(s)), and one or more adipogenic agents; when white adipocytes are used, the differentiation cocktail comprises one or more browning agents, and optionally, one or more adipogenic agents. Representative stem cells and progenitor cells include those derived from a mesenchymal or mesodermal lineage, as well as those capable of differentiating into such cells. In certain embodiments, the cells comprise adipose-derived stem cells, human embryonic stem cells, induced pluripotent stem cells, human bone marrow mesenchymal stem cells, preadipocytes, or progenitor cells found in adipose tissue or in skeletal muscle. If white adipocytes are used, the differentiation cocktail would consist of a browning agent(s) and optionally an adipogenic agent(s).

In embodiments in which the differentiation cocktail comprises one or more adipogenic agent(s), the adipogenic agent(s) can comprise one or more agents such as insulin, a glucocorticoid or synthetic equivalent (e.g., dexamethasone), cAMP enhancers such as indomethacin and 3-isobutyl-1-methylxanthine (IBMX), and vitamin C.

The browning agent can comprise a macromolecular crowder(s) and optionally can also include one or more of the following: a thyroid hormone (e.g. triiodothyronine), a PPARγ receptor agonist, a bone morphogenetic protein (e.g. BMP7), a retinoid (e.g. retinoic acid), a cardiac natriuretic peptide, a myokine (e.g. irisin), a fibroblast growth factor (e.g. FGF 21, FGF 2), a microRNA (e.g. mir193b-165), a lactogen (e.g. prolactin), an insulin-like growth factor (e.g. IGF-2), orexin, a bile acid, nitric oxide, a hyperacetylating agent, a hypomethylating agent, a prostaglandin, a PPARα ligand, TLQP-21, brain-derived neurotrophic factor, leptin, a β-adrenergic agonist, an AMPK activator, capsaicin or an analog thereof, fucoxanthin, 2-hydroxyoleic acid, resveratrol, conjugated linoleic acid, an n-3 fatty acid of marine origin, scallop shell powder (organic phase) and/or bofutsushosan. In a particular embodiment, the browning agent comprises a PPARγ receptor agonist that is a thiazolidinedione selected from the group comprised of rosiglitazone, ciglitazone, pioglitazone, darglitazone and troglitazone. In another particular embodiment, the browning agent is rosiglitazone or triiodothyronine (T3).

Macromolecular crowders can include one or more organic-based hydrophilic macromolecules (e.g., carbohydrate-based macromolecules), such as polymers of glucose and/or sucrose. If desired, the organic-based hydrophilic macromolecules can be neutral or derivatised (sulfated, acetylated, methylated) glucans; fructans; levans; glycosaminoglycans; and/or mixtures thereof.

In certain embodiments, the macromolecular crowders can comprise: an organic-based hydrophilic macromolecule having a molecular weight of 50 kDa to 500 kDa and a neutral surface charge; an organic-based hydrophilic macromolecule having a hydrodynamic radius range of 2 to 50 nm and a neutral or negative surface charge; or a mixture thereof. In other embodiments, the macromolecular crowders can comprise a mixture of at least two types of organic-based hydrophilic macromolecules, each type having a molecular weight of 50 kDa to 500 kDa and a neutral surface charge; a mixture of at least two types of organic-based hydrophilic macromolecules, each type having a molecular weight of 50 kDa to 500 kDa and neutral surface charge, together with a third type of organic-based hydrophilic macromolecule having a radius range of 2 to 50 nm and a neutral surface charge; and/or a mixture of at least two types of organic-based hydrophilic macromolecules, each type having a molecular weight of 50 kDA to 500 kDa and neutral surface charge, together with a third type of organic-based hydrophilic macromolecule having a radius range of 2 to 50 nm and a negative surface charge.

The functionality of the human brown adipocytes generated by the methods described herein can be verified by stimulating the adipocytes (e.g., with a specific β-adrenergic receptor agonist such as isoprenaline, noradrenalin, adrenalin, dobutamine, terbutaline, compound CL316243; or isoproterenol; or with a compound which elevates intracellular levels of cAMP such as dibutyryl-cAMP, 8-CPT-cAMP, 8-bromo-cAMP, dioctanoyl-cAMP, indomethacin, IBMX, or forskolin), and then quantifying one or more activities, such as expression of gene/protein, mitochondrial biogenesis, oxygen consumption, uncoupled respiration, glucose uptake, lipolysis, fuel metabolism or any other parameter which indicates increased metabolic activity, heat generation or other characteristics of adipocytes, and verifying that the characteristics of the human brown adipocytes are within desired parameters.

The invention further pertains to populations of human brown adipocytes prepared by such methods. The populations can be used, for example, as a screening platform to identify agents useful for altering metabolic activity of an individual (e.g., by promoting white to brown adipocyte transdifferentiation, or by promoting stem cell or progenitor cell differentiation into brown adipocytes), as well as for autologous cell-based therapies and methods for generating functional human brown adipocytes in an individual. In addition, the differentiation cocktails described herein can be used in pharmaceutical compositions. e.g., for biomaterials impregnated with a differentiation cocktail or for other including biomaterials, hydrogels, electrospun mesh, nano- or micro-particles, useful for generating brown adipocytes in an individual.

### BRIEF DESCRIPTION OF THE DRAWINGS

FIG. 1 depicts the activation of brown adipose tissue in humans. Stimulation of β3-adrenergic receptors leads to a dramatic increase in the intracellular concentration of triiodothyronine (T3) by means of the type 2 5′ deiodinase (DIO2); T3 in turn stimulates the transcription of uncoupling protein 1 (UCP1), which causes the leakage of protons from the inner membrane of the mitochondria, hence dissipating energy in the form of heat. cAMP=cyclic adenosine mono-phosphate, CRE=cAMP response element, TRE=thyroid hormone response element. Fig. from Celi F. N Engl J Med. 2009.

FIG. 2 depicts the distribution of brown adipose tissue (BAT) in human newborns, in comparison to that in adults. 2(A) BAT in infants is located in the interscapular, perirenal, mediastinal and in the neck region above and below the clavicles. 2(B) Schematic of BAT in cold-challenged adults via FDG-PET highlighting areas of high glucose uptake, a method originally used to detect tumors. Figure from Nedergaard et al. Am J Physiol Endocrinol Metab. 2007.

FIG. 3 depicts ELISA analysis of salt eluates of decellularised matrices, that indicates MMC increases the amount of FGF2 sequestered into the matrix by 30-fold, though no changes were evident with IGF1. (n=3; error bars are ±s.d.; ** P<0.01).

FIG. 4 demonstrates that adipocyte-derived ECM induces MSCs to express epigenetic markers of adipogenesis. ECM was deposited by adipogenically differentiated MSCs (adip) in the absence (−) and presence (+) of macromolecular crowding (MMC); These matrices where then decellularised and fresh undifferentiated MSCs seeded on them. Sequenome analysis of CpG methylation of two loci on the gene PDRM 16, revealed a similar methylation pattern occurring in cells that had been cultured on adipocyte matrices compared to those chemically induced (n=3; error bars are ±s.d.; ** P<0.01).

FIG. 5 indicates that macromolecular crowding alone can stimulate UCP1 mRNA expression in adipogenically induced mesenchymal stromal cells (MSCs). 5(a): Crowding with a white induction protocol (Iw+MMC) already induces a 10-fold increase of UCP1; while together with a brown induction protocol (Ib+MMC) UCP1 expression is increased by 23-fold. A brown induction protocol alone without macromolecular crowder did not significantly increase the UCP1 expression. Note that BMP7 (Ib group) only modestly increases UCP1 expression compared to the Ib (−BMP7) group, indicating that BMP7 may not be critical in the differentiation process (n=3; error bars are ±s.e.; * P<0.05 ** P<0.01).

FIG. 6 indicates that macromolecular crowding under a brown induction protocol induces massive upregulation of thermogenic genes after 4 hrs of forskolin stimulation. Compared to a classical white induction protocol without MMC and forskolin stimulation. 6(A) UCP1 mRNA expression is increased by several hundredfold. 6(B) PGC-1α 35 times (C) DIO2 6 times (compare with FIG. 1) (n=3; error bars are ±s.e.; * P<0.05 ** P<0.01).

FIG. 7 depicts results of forskolin treatment of white and brown adipogenically induced mesenchymal stem cells, that leads to emptying of lipid deposits. Nile Red content of MSCs as quantified using a bioimaging station shows Nile Red positive areas expressed as μm² and normalised for cell numbers. Conditions: C=non-induced control; Iw=white induction protocol; Ib=brown induction protocol (n=3; error bars are ±s.e.; ** P<0.01).

FIG. 8 indicates that isoproterenol treatment of white and brown adipogenically induced mesenchymal stem cells leads to emptying of lipid droplets. Isoproterenol exerted a stronger lipolytic effect on adipocytes that had been differentiated under a BAT induction protocol. (n=3; error bars are ±s.e.; * P<0.05).

FIG. 9 demonstrates that macromolecular crowding promotes a white-to-brown conversion of MSC-derived adipocytes. MSCs were induced to differentiate for 3 weeks into white adipocytes using the standard white induction protocol (Iw), then induced for the next 3 weeks with the brown induction protocol±MMC (Ib or Ib mmc). Exposing mature white adipocytes (at 3 weeks) to the brown induction protocol with crowding for 3 more weeks (week 0-3: Iw; week 4-6: Ib mmc) showed a 35.7-fold upregulation of UCP1 compared to just the brown induction protocol alone (week 0-3: Iw; week 4-6: Ib), which only had a 6.5-fold upregulation of UCP1 (n=2; error bars are ±s.e.; ** P <0.01).

FIG. 10 depicts a representative timeline for adipogenic differentiation.

### DETAILED DESCRIPTION OF THE INVENTION

The present invention relates to methods of generating functional human brown adipocytes from human stem cells or progenitor cells, such as from mesenchymal progenitor/stem/stromal cells, using a differentiation cocktail that comprises one or more adipogenic agents and one or more browning agents (e.g., one or more macromolecular crowders), as well as to a differentiation cocktail as described herein. This invention also relates to methods of transdifferentiation of white adipocytes into brown adipocytes by using a differentiation cocktail that comprises one or more browning agents (e.g., one or more macromolecular crowders), and optionally one or more adipogenic agents, as well as to such differentiation cocktails

A characteristic of the microenvironment of all cells is the high total concentration of macromolecules. Such media are termed ‘crowded’ rather than ‘concentrated’ because, in general, no single macromolecular species occurs at high concentration but, taken together, account for a volume occupancy of 20-30% of a given specific volume. As pointed out by Ellis (Ellis, R J, Trends Biochem Sci, 26(10):597-604 (2001)), and Minton (Minton, A P, Curr Biol, 10(3):R97-9 (2000)), crowding by macromolecules has both thermodynamic and kinetic effects on the properties of other macromolecules that are not generally appreciated. Biological macromolecules such as enzymes have evolved to function inside such crowded environments. For example, the total concentration of protein and RNA inside bacteria like E. coli is in the range of 300-400 g/l. Macromolecular crowding causes an excluded volume effect (EVE), because the most basic characteristic of crowding agents is the mutual impenetrability of all solute molecules. This nonspecific steric repulsion is always present, regardless of any other attractive or repulsive interactions that might occur between the solute molecules. Thus, crowding is an inevitable hallmark of the intracellular milieu of all carbon-based life-forms on earth (reviewed in Ellis, R J, Trends Biochem Sci, 26(10):597-604 (2001)). The effects resulting from macromolecular crowding are so large that authorities in the field state that many estimates of enzyme catalyzed reaction rates and equilibria made with uncrowded solutions in the test tube differ by orders of magnitude from those of the same reactions operating under crowded conditions within cells (Ellis, R J, Trends Biochem Sci, 26(10):597-604 2001).

Despite this knowledge, biochemists still commonly study enzymatic reactions in solutions with a total macromolecular concentration of 1-10 g/l or less, in which crowding is negligible. A particular example is the polymerase chain reaction which is performed in a diluted aqueous environment. If crowdedness in introduced into such a system emulating an intracellular environment, the kinetics shift dramatically, the reaction is accelerated, more amplicons are generated, the enzyme is heat-protected, and primer-template interactions are enhanced (Lareu, R R, et al., Biophy Biochem Res Comm, 363(1):171-177 (2007c), Harve, K S, et al., Nucleic Acids Res, epub (Oct. 23, 2009), Raghunath, M et al. WO 2008/018839 A1, all of which are herein incorporated by reference).

The principle of macromolecular crowding also reigns in the extracellular environment. Cells are surrounded by soluble and immobilised macromolecules which form their native microenvironment. Again, contemporary cell culture consists of placing adhering cells on a support (tissue culture plastic or other materials) or keeping them in suspension in aqueous media under conditions that do not reflect the crowded environment from which they have been originally derived. Thus, they cannot exert they physiological function to the fullest potential. In fact, it has been shown that when fibrogenic cells are grown under crowded conditions using negatively charged crowders, enzymatic steps are accelerated that control the deposition rate of collagen (Lareu, R R., et al., Tissue Engineering, 13(2):385-391 (2007a); Lareu, R R., et al., FEBS Lett, 581(14):2709-2714 (2007b)).

In the methods of the invention, human stem cells, progenitor cells, or human white adipocytes are used. The progenitor cells or stem cells can include, for example, cells derived from (descended from) a mesenchymal or mesodermal lineage, as well as from cells that are capable of differentiating into cells of mesenchymal or mesodermal lineage. Representative stem cells or progenitor cells include adipose-derived stem cells, human embryonic stem cells (HES), induced pluripotent stem cells (iPS), human bone marrow mesenchymal stem cells (hbmMSCs), preadipocytes, and progenitor cells found in adipose tissue or in skeletal muscle.

The stem cells, progenitor cells, or white adipocytes are subjected to a differentiation cocktail that comprises a browning agent such as a macromolecular crowder. A “browning agent,” as used herein, refers to an agent that facilitates transformation of the stem cells, progenitor cells, or white adipocytes to brown adipocytes by driving adipogenesis towards a brown lineage. In particular embodiments, the browning agent comprises a macromolecular crowder(s) as described below. In certain embodiments, the differentiation cocktail optionally additionally includes one or more of the following browning agent(s): a thyroid hormone (e.g. triiodothyronine), a PPARγ receptor agonist, a bone morphogenetic protein (e.g. BMP7), a retinoid (e.g. retinoic acid), a cardiac natriuretic peptide, a myokine (e.g. irisin), a fibroblast growth factor (e.g. FGF 21, FGF 2), a microRNA (e.g. mir193b-165), a lactogen (e.g. prolactin), an insulin-like growth factor (e.g. IGF-2), orexin, a bile acid, nitric oxide, a hyperacetylating agent, hypomethylating agent, a prostaglandin, a PPARα ligand, TLQP-21, brain-derived neurotrophic factor, leptin, a β-adrenergic agonist, an AMPK activator, capaisin and its analogs, fucoxanthin, 2-hydroxyoleic acid, resveratrol, conjugated linoleic acid, an n-3 fatty acid of marine origin, scallop shell powder (organic phase) and/or bofutsushosan. In a particular embodiment, the browning agent comprises a PPARγ receptor agonist that is a thiazolidinedione selected from the group comprised of rosiglitazone, ciglitazone, pioglitazone, darglitazone and troglitazone. In another particular embodiment, the browning agent is rosiglitazone and/or triiodothyronine (T3).

“Macromolecular crowding,” as used herein, refers to culturing in the presence of macromolecular crowders. In the methods of the invention, the differentiation cocktail comprises one or more browning agents such as one or more organic-based hydrophilic macromolecules, also referred to herein as a crowder macromolecule(s) or a macromolecular crowder. In another embodiment, two or more (at least two) carbohydrate-based macromolecules are used. In particular aspects, multiple, e.g., two, three, or four, etc. organic-based hydrophilic macromolecules are used.

As used herein “macromolecular crowders” are inert or nontoxic macromolecules and can be of any shape (e.g., spherical shape), and are typically of neutral or negative surface charge with a molecular weight above about 50 kDa (see WO 2011/108993, which is herein incorporated by reference). In a particular aspect, the macromolecules are carbohydrate based. Representative macromolecules according to the invention may have a molecular weight of from about 50 kDa to about 1000 kDa. In specific aspects, the molecular weight of the macromolecule is about 50, 100, 200, 300, 400, 500, 600, 700, 800, 900, or 1000 kDa. In a particular aspect, the organic-based macromolecule according to the invention is a carbohydrate-based hydrophilic macromolecule. For example, the carbohydrate-based macromolecule of the invention may be a polymer of glucose and/or sucrose. Particular examples of the macromolecule according to the invention include Ficoll™ 70, Ficoll™ 400, polyvinyl pyrrolidone (PVP), glycosaminoglycans, sugar chains of glycosaminoclycans, cellulose, pullulan or a mixture thereof. Specifically, the carbohydrate-based macromolecule can be Ficoll™ 70, Ficoll™ 400, dextran, neutral dextran (neutral dextran 410; neutral dextran 670, PVP 360 kDa, pullulan, dextran sulfate, polystyrene sulfonate, chondroitin sulfate, heparin sulfate, heparan sulfate, dermatan sulfate or a mixture thereof. In particular aspects, the carbohydrate-based hydrophilic macromolecule is Ficoll. In yet another aspect, the macromolecular crowder used in the method is a mixture of Ficoll™ 70 and Ficoll™ 400. Ficoll can be obtained from commercial sources such as GE Healthcare as Ficoll™ 70 (Fc70; 70kDa) under catalogue number 17-0310 and Ficoll™ 400 (Fc400; 400kDa) under catalogue number 17-0300.

In other aspects of the methods provided herein, the differentiation cocktail comprising macromolecule(s) as browning agents may have a viscosity of less than about 2 mPa-s. For example, a viscosity of about 1.75 mPa-s, 1.5 mPa-s, 1.25 mPa-s, 1mPa-s 0.75 mPa-s, 0.5 mPa-s, or 0.25 mPa-s.

In yet other aspects, the macromolecules can have a hydrodynamic radius range of from about 2 nm to about 50 nm, from about 5 nm to about 20 nm or from about 10 nm to about 15 nm.

In some aspects, the total macromolecular concentration is about 2.5-100 mg/ml, and in other aspects, about 5-90 mg/ml, about 10-80 mg/ml, about 20-70 mg/ml, about 30-60 mg/ml, about 40-50 mg/ml, and in yet other aspects about 10-40 mg/ml, about 10-62.5 mg/ml, or about 10-37.5 mg/ml. In particular aspects, the macromolecule may be Ficoll™ 70 present at a concentration of 2.5-100 mg/ml, and/or Ficoll™ 400 at a concentration of 2.5-100 mg/ml, or a mixture thereof. In other particular aspects, the macromolecule may be Ficoll™ 70 present at a concentration of 2.5-37.5 mg/ml and/or Ficoll™ 400 at a concentration of 2.5-25 mg/ml, or a mixture thereof. In a particular aspect, the stem cells are contacted with a carbohydrate-based macromolecule comprising Ficoll™ 70 at a concentration of about 37.5 mg/ml and Ficoll™ 400 at a concentration of about 25 mg/ml.

The concentration of macromolecules for use in the present invention can also be calculated based on the volume fraction occupancy. As known to those of skill in the art, the composition of a solution containing very large molecules (macromolecules) such as polymers, is most conveniently expressed by the “volume fraction (Ψ)” or “volume fraction occupancy” which is the volume of polymer used to prepare the solution divided by the sum of that volume of macromolecule and the volume of the solvent. In the methods described herein the cells are contacted with the one or more macromolecules at a biologically relevant volume fraction occupancy. In some aspect, the biologically relevant volume fraction occupancy is from about 3% to about 30%. In other aspects, the biologically relevant volume fraction occupancy is from about 5% to about 25%, from about 10% to about 20% and from about 12% to about 15%. Thus, in the methods of the invention, the volume fraction occupancy is about 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29% or 30%. In a particular aspect, the biologically relevant volume fraction occupancy is about 15%.

One or more type(s) of macromolecular crowder(s) can be used, and combinations of various sizes and types of surface charge (e.g., neutral or negative) can be employed. In certain embodiments, one or more of the macromolecules has a radius range of 2 to 50 nm; in certain other embodiments, one or more of the macromolecules has a molecular weight of 50 kDa to 1000 kDa (e.g., 50 kDa to 500 kDa). Additionally, if desired, one (or more, if used) of the types of organic-based hydrophilic macromolecules is a carbohydrate-based hydrophilic macromolecule. Representative macromolecules include, for example, polymers of glucose and/or sucrose. If desired, at least one of the types of organic-based hydrophilic macromolecules can be neutral or derivatised (sulfated, acetylated, methylated) glucans; fructans; levans; or glycosaminoglycans.

For example, in certain embodiments, the molecular crowder(s) can comprise: (a) an organic-based hydrophilic macromolecule having a molecular weight of 50 kDa to 1000 kDa (e.g., a molecular weight of 50 kDa to 500 kDa) and neutral surface charge; (b) an organic-based hydrophilic macromolecule having a radius range of 2 to 50 nm and neutral or negative surface change; or (c) a combination of such macromolecules. In other aspects, the method can comprise using two or more of such organic-based hydrophilic macromolecules, each having neutral surface change.

In other representative embodiments, the method comprises using for the browning agent: macromolecular crowders that comprise (a) two or more organic-based hydrophilic macromolecules, each having a molecular weight of 50 kDa to 1000 kDa (e.g., a molecular weight of 50 kDa to 500 kDa) and neutral surface charge, or (b) two or more organic-based hydrophilic macromolecules, each having a radius range of 2 to 50 nm and neutral or negative surface change, or (c) two or more organic-based hydrophilic macromolecules each having a molecular weight of 50 kDa to 1000 kDa (e.g., a molecular weight of 50 kDa to 500 kDa) and a neutral surface charge, combined with a third organic-based hydrophilic macromolecule having a molecular weight of 50 kDa to 1000 kDa (e.g., a molecular weight of 50 kDa to 500 kDa) and a neutral surface charge, or (d) two or more organic-based hydrophilic macromolecules each having a molecular weight of 50 kDa to 1000 kDa (e.g., a molecular weight of 50 kDa to 500 kDa) and neutral surface charge, combined with a third organic-based hydrophilic macromolecule having a molecular weight of 50 kDa to 1000 kDa (e.g., a molecular weight of 50 kDa to 500 kDa) and having a negative or neutral surface charge; (e) a mixture of two or more of organic-based hydrophilic macromolecules, each type having a molecular weight of 50 kDa to 500 kDa and neutral surface charge, together with a third type of organic-based hydrophilic macromolecule having a radius range of 2 to 50 nm and a neutral surface charge; or (f) a mixture of two or more types of organic-based hydrophilic macromolecules, each type having a molecular weight of 50 kDa to 1000 kDa (e.g., a molecular weight of 50 kDa to 500 kDa) and neutral surface charge, together with a third type of organic-based hydrophilic macromolecule having a radius range of 2 to 50 nm and a negative surface charge.

Representative macromolecules include, for example, Ficoll™ 70, Ficoll™ 400, dextran, neutral dextran (e.g. neutral dextran 410 kDa, neutral dextran 670 kDa), pullulan, dextran sulfate, cellulose, amylose, glycogen, chondroitin sulfate, heparan sulfate, heparin, heparin sulfate, dermatan sulfate, hyaluronic acid, and starch. Mixtures thereof can be used as well, if desired. In a particular embodiment, a mixture of Ficoll™ 70 and Ficoll™ 400 is used for the mixture of macromolecules. The concentration of the macromolecules can be varied; in one embodiment, the concentration is about 2.5-100 mg/ml. If Ficoll™ 70 and Ficoll™ 400 are used, for example, Ficoll™ 70 can be present at a concentration of about 7.5-100 mg/ml (e.g., 25-50 mg/ml, such as 37.5 mg/ml), and Ficoll™ 40 can be present at a concentration of 2.5-100 mg/ml (e.g., 10-50 mg/ml, such as 25 mg/ml). Viscosity of the macromolecules can be varied; in certain embodiments, the macromolecules have a viscosity of less than 2 mPa·s.

As will be appreciated by those of skill in the art, additional macromolecular crowders can be added if desired. In one aspect, the additional crowder(s) is either a neutrally charged crowder (e.g., PVP) or a negatively charged crowder (e.g., Dextran sulfate 500 kDa) (e.g., see WO 2011/108993, which is herein incorporated by reference).

In the methods of the invention, if human stem cells or progenitor cells as described above are used, the cells are cultured with a differentiation cocktail comprising one or more adipogenic agent(s) as described below, and one or more browning agent(s) (e.g., macromolecular crowder(s)); if white adipocytes are used, the cells are cultured with a differentiation cocktail comprising one or more browning agent(s) (e.g., macromolecular crowder(s)), and optionally if desired, one or more adipogenic agent(s) as described below, When macromolecular crowder(s) are used as the browning agent, the macromolecules can be added to the cocktail in a variety of ways. For example, the macromolecules are added as a powder or liquid into culture medium. Preferably, the addition of the macromolecule does not significantly increase the viscosity of the cell culture medium. The medium can then be sterilized, e.g. via filtration, if desired. In one aspect, the crowders include a combination of Ficoll 70 and Ficoll 400.

“adipogenic agent,” as used herein, refers to one or more agents selected to facilitate adipogenesis. In representative embodiments, one or more adipogenic agents are selected from the group consisting of: insulin, a glucocorticoid or synthetic equivalent (e.g., dexamethasone), cAMP enhancers such as indomethacin and 3-isobutyl-1-methylxanthine (IBMX), and vitamin C.

Culturing the cells in the presence of the differentiation cocktail yields a population of functional human brown adipocytes. “Functional” human brown adipocytes, as used herein, refers to adipocytes that exhibit the characteristics of brown adipocytes. Characteristics include, for example, production of molecular markers or activities characteristic of brown adipocytes, such as expression of the UCP1 gene/protein and/or other representative brown fat associated gene(s)/protein(s); mitochondrial biogenesis; oxygen consumption; uncoupled respiration; glucose uptake; lipolysis; fuel metabolism or any other parameter which indicates increased metabolic activity and/or heat generation. For example, human brown adipocytes typically express representative brown fat genes/proteins, such as UCP1. Other representative brown fat genes/proteins include CIDEA, CPT1B, PRDM16, DIO2, PGC1α etc. Assessment of the expression of such genes in cells exposed to the differentiation cocktail can be performed and compared to the expression in brown adipocytes or, alternatively or in addition, to the expression in white adipocytes, in order to assess whether the cells display functional human brown adipocyte characteristics. In a particular embodiment, UCP1 expression is at a level that is significantly different than that of a human white adipocyte population.

In an aspect of the invention, the functionality of the human brown adipocytes can be assessed by examining activation of the thermogenic programme in these cells by one or more factors. Activation of thermogenic programme, as used herein, indicates the transcription pathway leading to the upregulation of UCP1 expression and activity in the mitochondria is activated, and consequently increased uncoupled respiration, increased mitochondrial respiration and subsequently heat generation occur. Current parameters used to measure this phenomenon include, for example, expression of the UCP1 gene/protein; mitochondrial biogenesis; oxygen consumption; uncoupled respiration; glucose uptake; lipolysis; fuel metabolism or any other parameter which indicates increased metabolic activity and/or heat generation.

The factors activating the thermogenic programme are for example stimulating the cells with a specific β-adrenergic receptor agonist (e.g., isoprenaline, noradrenalin, adrenalin, dobutamine, terbutaline, compound CL316243, or isoproterenol) and/or a compound which elevates intracellular levels of cAMP (e.g., dibutyryl-cAMP, 8-CPT-cAMP, 8-bromo-cAMP, dioctanoyl-cAMP, indomethacin, IBMX, or forskolin). Functionality of the human brown adipocytes is verified when the thermogenic programme is activated upon stimulation, i.e. the expression of the UCP1 gene/protein, and/or of the mitochondrial biogenesis, and/or of oxygen consumption, and/or of uncoupled respiration, and/or of glucose uptake and/or of lipolysis and/or of fuel metabolism and/or of any other parameter which indicates increased metabolic activity and/or heat generation, is increased compared with a comparable measurement obtained in the absence of simulation by the specific β-adrenergic receptor agonist and/or compound which elevates intracellular levels of cAMP.

The invention further pertains to populations of functional brown adipocytes prepared by the methods described herein. Such populations can be used, for example, in a screening platform to identify agents capable of altering the metabolic activity of an individual by activating the thermogenic programme of the brown adipocytes; by promoting white to brown adipocyte transdifferentiation; or by promoting differentiation of human stem cells or progenitor cells to brown adipocytes.

For example, a population of functional brown adipocytes can be exposed to an agent of interest, and then monitored to assess the effects of the agent on the brown adipocytes. In certain embodiments, methods such as that described above for assessing the functionality of the human brown adipocytes can be used (e.g., quantifying one or more molecular markers and activities characteristic of brown adipocytes, such as: expression of the UCP1 gene/protein; mitochondrial biogenesis; oxygen consumption; uncoupled respiration; glucose uptake; lipolysis; fuel metabolism or any other parameter which indicates increased metabolic activity; and/or heat generation of the human brown adipocytes). Agents that increase the expression, presence or activity of the marker or activity characteristic of the brown adipocytes (e.g., such as by activating the thermogenic programme of brown adipocytes), are identified as agents of interest that may be capable of altering metabolic activity in an individual.

Similarly, a population of functional brown adipocytes can be used as a positive control in an experiment to assess agents for their ability to promote differentiation of stem cells, progenitor cells, or white adipocytes. Stem cells, progenitor cells, or white adipocytes unexposed to an agent of interest would serve as a baseline; comparable stem cells, progenitor cells, or white adipocytes would then be exposed to an agent of interest, and assessed as described herein for brown adipocyte functionality. If the cells demonstrated similar functionality to a population of functional brown adipocytes as described herein, the agent of interest would be an agent that is capable of promoting differentiation of the relevant stem cells, progenitor cells, or white adipocytes to brown adipocytes.

The invention further pertains to use of the differentiation cocktails described herein, as an additive for cell cultures. For example, MSCs or other cells for generating brown adipocytes can be cultured in the presence of a differentiation cocktail (e.g., in a bioreactor setting or in a submerged monolayer culture). Such culture can yield brown adipocytes (e.g., in BAT monolayers) that can be used in metabolic, genetic, epigenetic, cell biological and/or calorimetric studies.

In yet another aspect, the invention is directed to a pharmaceutical composition comprising a differentiation cocktail as described herein. The differentiation cocktail described herein can be formulated with a physiologically acceptable carrier or excipient to prepare a pharmaceutical composition. The carrier and composition can be sterile. The formulation should suit the mode of administration.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like that do not deleteriously react with the active compounds.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc.

The pharmaceutical compositions thereof can be administered systemically and/or locally. Methods of introduction of these compositions include, but are not limited to, intradermal, intramuscular, intraperitoneal, intraocular, intravenous, subcutaneous, topical, oral and intranasal. Other suitable methods of introduction can also include gene therapy, rechargeable or biodegradable devices, particle acceleration devises (“gene guns”) and slow release polymeric devices. For example, hydrogel cultures in a 3D in vitro bioreactor can be employed in the presence of differentiation cocktails, such as to generate an implantable composition that comprises brown adipocyte tissue or layers of brown adipocytes (e.g., implantable cell sheets). In other examples, differentiation cocktails can be incorporated into biomaterials, into injectable hydrogels, into microparticles, or otherwise packaged to be administered to a human individual (e.g., into fat deposits such as subcutaneously, or otherwise administered into fat tissue or muscle).

The pharmaceutical compositions of this invention can also be administered as part of a combinatorial therapy with other compounds.

The composition can be formulated in accordance with the routine procedures as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active compound. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., that are, if desired, sterilized or mixed with auxiliary agents, e.g., preservatives, stabilizers, wetting agents, buffers or salts for influencing osmotic pressure, etc. The compound may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

Compounds described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

For example, the differentiation cocktail as described herein can be incorporated into a pharmaceutical composition for administering to the individual. Representative pharmaceutical compositions comprise biomaterials permeated or impregnated with the differentiation cocktail; for example, biodegradable biomaterials, or biomaterials with degradable coatings, can be administered (e.g., implanted) as a film, tube, mesh (e.g., electrospun mesh/mat/fibers), foam, granules, or other form, either in single structure or particle form. In one particular embodiment, hydrogels (e.g., containing collagen, hyaluronic acid etc.), nano- or micro-particles (e.g., coated with the differentiation cocktail or having it incorporated therein), can be used. Other pharmaceutical compositions can comprise pharmaceutical solutions for injection, either alone or in combination with another biomaterial (e.g., a hydrogel, nano- or micro-particles), or to be delivered by dendrimer technology. In particular embodiments, such pharmaceutical compositions can be administered by direct injection, ballistic or biolistic application (particle or gene gun or powder jet), or implantation (e.g., into adipose tissue or another region), as well as other appropriate means of delivery to a human individual.

The invention additionally pertains to autologous cell-based ex vivo therapy, as well as pharmacological in vivo therapy, using the methods, differentiation cocktails, pharmaceutical compositions, and/or populations of functional brown adipocytes, as described herein. For example, for autologous therapy, a sample of stem cells or progenitor cells can be obtained from a human individual, and then the methods described herein can employed to generate functional brown adipocytes. Such adipocytes can then be returned to the same human individual, thereby introducing the brown fat cells or brown fat tissue into the individual. In another embodiment, the differentiation cocktails can be incorporated into electorpsun fibers, and such fibers can be implanted with MSCs in vivo for autologous therapy. In a further embodiment, differentiation cocktails can be incorporated into various biomaterials (e.g., materials as described above) and implanted into white fat deposits in a human individual (e.g., subcutaneously), or otherwise administered into fat tissue or other tissue (e.g., muscle). Such use can promote white to brown adipocyte transdifferentiation in the individual. For pharmacological therapy, a differentiation cocktail or a pharmaceutical composition comprising the differentiation cocktail can be administered to a human individual, thereby generating additional brown adipocytes in the individual.

Both such ex vivo and in vivo methods can be employed for weight reduction therapy for the treatment of obesity and its related diseases such as metabolic syndrome, diabetes, atherosclerosis, cardiovascular heart disease, hypertension, stroke, osteoarthritis and some cancers (breast, colon) (see, e.g., for related diseases, the WHO Factsheet: Obesity and Overweight.

http://www.who.int/mediacentre/factsheets/fs311/en/index.html#(2011)).

### DISCUSSION

Brown adipocytes and general methods of the invention: Brown adipose tissue is currently believed to hold the key for energy consumption and weight control of an individual. Brown adipocytes have a huge capacity for triglyceride clearance and glucose disposal. A unique feature is their ability to perform uncoupled mitochondrial respiration due to the presence of uncoupling protein. UCP1. Thus, brown adipocytes are producers of heat and are solely responsible for non-shivering thermogenesis. Originally believed to be present only in newborns, brown adipocytes have been recently discovered in circumscribed locations in adults. This has inspired clinical researchers to investigate brown adipocytes as therapeutic targets for treating obesity, diabetes, and metabolic syndrome; however, no source for in vitro cultivation of human brown adipocytes previously has existed. Investigations into the developmental origin and function of brown adipose tissue have been done in mice and murine precursor cell lines.

We have recently developed proprietary macromolecular crowding technology to boost white adipogenic differentiation of human bone marrow mesenchymal stem cells [see PCT/SG2011/000081; WO2011/108993 A1]. We have now discovered, that, surprisingly, this culture system increases basal levels of UCP1 gene expression 10-fold already during a white induction protocol. This means that mixed macromolecular crowding alone with polysucrose can induce a ‘browning’ effect. Using this system (with, for example, a mixture of Ficoll™ 70 and Ficoll™ 400) under conditions of brown fat induction, there is a several 100-fold upregulation upon forskolin stimulation. We therefore can generate human brown adipocytes from other cells, including from white adipocytes. This invention is the basis for a pharmacological platform and cell-based therapy of obesity and metabolic syndrome.

Obesity issues and relationship to brown and white adipocytes: Obesity develops when energy intake exceeds energy expenditure. In developed countries we see now a combination of an ample and cheap supply of processed food, and meat products. In combination with sedentary lifestyle, and the inability of the CNS to suppress appetite appropriately, these lead to an energy imbalance and the passive storage of excessive calories in adipose tissue. However, adipose tissue is not only a fat store but also an active endocrine organ, releasing free fatty acids and adipokines such as leptin, adiponectin, TNFα, interleukin-6, and retinol binding protein-4, all of which can act on other tissues, including the brain, liver, and muscle to regulate food intake, energy balance, and insulin sensitivity (Cypess, A. M. et al. Current Opinion in Endocrinology, Diabetes and Obesity, 17, 143-149 (2010)).

White adipose tissue (WAT) distribution (intestinal vs subcutaneous) greatly affects metabolic risk. In addition to WAT, which stores energy, mammals including humans have brown adipose tissue (BAT), which burns energy for thermogenesis. In small mammals BAT is important for thermogenesis and energy balance. BAT induction in mice promotes energy expenditure, reduces adiposity, and protects from diet-induced obesity. BAT ablation reduces energy expenditure and increases obesity in response to high-fat diets [reviewed in Cypess & Khan 2010]. Brown adipocytes display numerous, large mitochondria. The inner mitochondrial membrane carries the BAT-specific uncoupling protein 1 (UCP1), which when activated dissipates the intermembrane proton-motive force and generates heat instead of ATP. It was estimated earlier that in humans as little as 50 g of BAT could utilize up to 20% of basal caloric needs if maximally stimulated (Rothwell, N. J. et al. Clin Sci (Lond). 64, 19-23 (1983).)

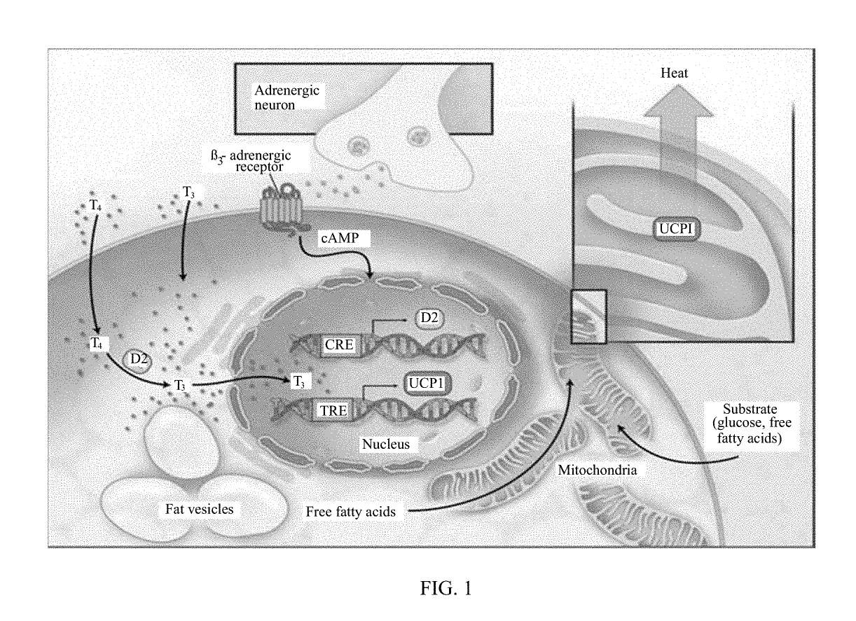

FIG. 1 depicts the activation and activity of brown adipose tissue. Stimulation of β3-adrenergic receptors leads to a dramatic increase in the intracellular concentration of triiodothyronine (T3) by means of the type 2 5′ deiodinase (DIO2); T3 in turn stimulates the transcription of uncoupling protein 1 (UCP1), which causes the leakage of protons from the inner membrane of the mitochondria, hence dissipating energy in the form of heat. cAMP=cyclic adenosine mono-phosphate, CRE=cAMP response element, TRE=thyroid hormone response element (From Celi F. N Engl J Med. 2009).

UCP1 is the signature protein of BAT and is necessary to mediate thermogenesis. In addition, to UCP1, brown adipocytes can be distinguished from WAT at the molecular level by high-levels of expression of type 2 iodothyronine deiodinase (DIO2 in FIG. 1) and the transcription co-regulator such as PGC-11α. (Gesta, S., et al. Cell. 131, 242-256 (2007)).

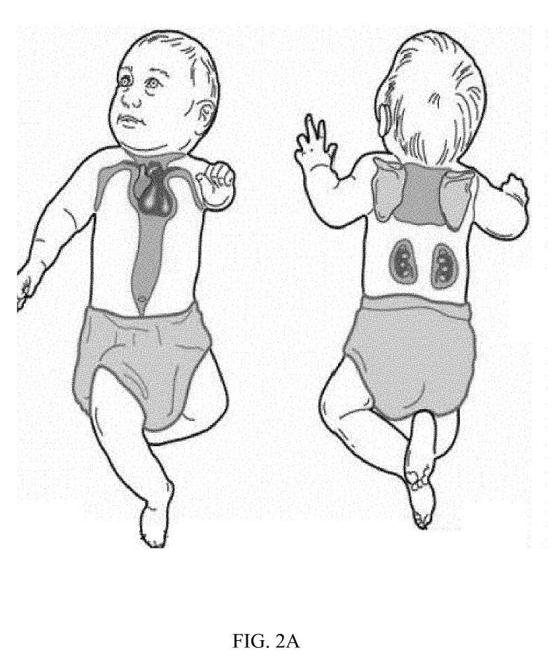

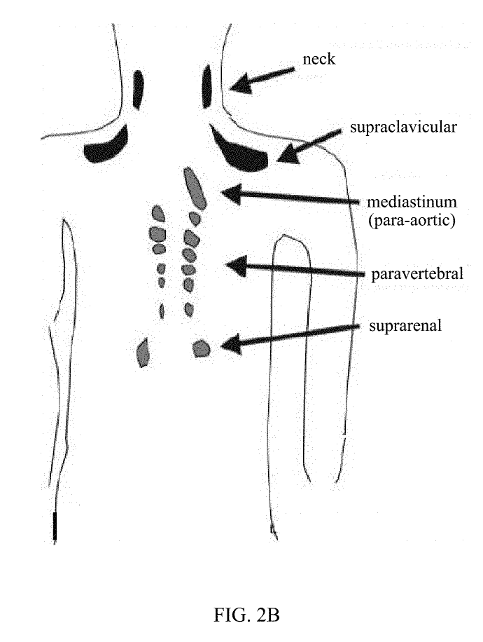

The (re)discovery of brown fat in adult humans: BAT is crucial for metabolic regulation in rodents, and was known to exist in human newborns in certain locations (FIG. 2). However, as BAT deposits were difficult to detect histologically in adults, the importance and function of BAT in normal adult humans was considered biologically irrelevant in adult humans. With the advent of positron-emission tomography in combination with computed tomography (PET/CT) to search for metabolically active tumours using 18F-fluorodeoxyglucose (18F-FDG), radiologists noted small, but distinct, non-tumor collections of adipose tissue with high uptake of this tracer. In 2007 the first unexpected finding of BAT in adults was made (Nedergaard, J., et al. AJP: Endocrinology and Metabolism. 293, E444-E452 (2007)), and in 2009 five independent groups showed presence and relevance of BAT in adult humans using 18F-FDG PET/CT (Saito, M. et al. Diabetes. 58, 1526-1531 (2009); Zingaretti, M. C. et al. FASEB J. 23, 3113-3120 (2009); van Marken Lichtenbelt, W. et al. N Engl J Med. 360, 1500-1508 (2009); Cypess, A. et al. N Engl J Med. 360, 1509-1517 (2009); Virtanen, K. A. et al. N Engl J Med. 360, 1518-1525 (2009).

These depots of metabolically active fat were found in the cervico-supraclavicular region, contained UCP1 and displayed a BAT histology. (FIG. 2).

FIG. 2 describes the distribution of BAT in newborns, infants in comparison to that in adults. (A) BAT in infants is located in the interscapular region, perirenal, mediastinal in the neck region above and below the clavicles. (B) Schematic of BAT in cold-challenged adults via FDG-PET highlighting areas of high glucose uptake, a method originally used to detect tumors.

Advantages of the invention—availability of human brown adipocytes for research and clinical application: Adipose tissue is a major endocrine and secretory organ in humans. Yet, current models of adipogenic cell differentiation and functionality are based on immortalized lines such as 3T3 L1, a murine preadipocyte cell line. The present invention, which utilizes human primary and multipotent cells and stem cells, is much more clinically relevant. This invention allows the construction of a BAT screening platform to identify compounds to counter obesity, diabetes and other metabolic diseases, for nutrition and nutraceuticals, as well as the pharmaceutical industries. Also, this invention provides a platform to convert autologous MSCs into BAT for re-implantation purposes to drive the metabolic rate up and as auxiliary treatment of metabolic syndrome.

Advantages of the invention—pharmacological conversion of white adipocytes/preadipocytes to brown adipocytes: Thiazolidinediones, a class of insulin-sensitizing drugs, are used to treat type 2 diabetes through selectively activating PPARγ (Yki-Järvinen, H. Thiazolidinediones. N Engl J Med. 351, 1106-1118 (2004)). Interestingly, this drug class seems to exert a “browning” effect on white adipocytes, pushing them to express UCP1 and other thermogenic markers. (Bogacka et al. Diabetes. 54, 1392-1399 (2005)) showed that pioglitazone induced mitochondrial biogenesis in human subcutaneous adipose tissue in vivo of type 2 diabetic patients along with upregulated expression of thermogenic genes such as PGC-1α and UCP1. Recently (Vernochet et al., Mol Cell Biol. 29, 4714-4728 (2009)) demonstrated that troglitazone upregulated ucpl and other BAT genes in mature 3T3-L1 white adipocytes. (Petrovic et al., J Biol Chem. 285, 7153-7164 (2010)) demonstrated that chronic treatment of rosiglitazone in precursor cells from the epididymal region of mice promotes the expression of the thermogenic programme, including norepinephrene-inducible Ucp1 gene. Thus, with the platform we have developed a tool to convert WAT to BAT in vitro, this is on account of the “browning” effect described above using rosiglitazone.

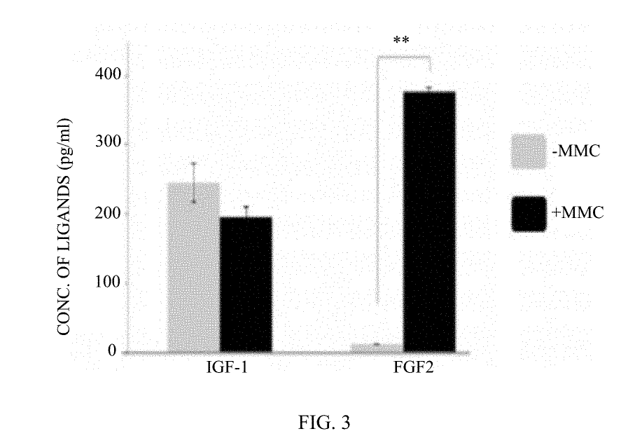

Macromolecular crowding (MMC) enhances deposition and remodeling of the extracellular matrix (ECM) and increases the amount of FGF2 sequestered in the matrix. The ECM as a component of the microenvironment plays an important role in directing the differentiation and maintaining cellular phenotype. Thus, we examined whether MMC affected the deposition and remodeling of the ECM proteins specifically involved in adipogenesis. We observed morphological transition of the ECM in the course of differentiation from a longitudinal-reticulate pattern (fibronectin and collagen IV) to a honey comb pattern; in parallel we observed an increased degradation of fibronectin as has been described for adipogenic matrix remodeling in murine preadipocyte 3T3 cells. We also confirmed that the enhanced ECM deposition was accompanied by an increase of matrix-bound heparin sulfate-rich proteoglycans. Sulfated polysaccharides such as heparin and heparan play an important role of sequestering a variety of growth factors including fibroblast growth factor (FGF), transforming growth factor, bone morphogenic proteins (BMP) and hepatic growth factor. In fact, in good correlation with immunocytochemical observation of a stronger presence of heparan sulfate in the ECM we found a 30-fold increase in the amount of FGF2 sequestered in the cell layer of adipogenically induced MSCs in the presence of MMC. There was no significant difference in the amount of IGF1 sequestered in the adipogenically induced cell layers +/−MMC (FIG. 3), while BMP4 was not detectable (data not shown).

Previous experiments indicated that adipogenically induced MSCs under macromolecular crowding deposited more extracellular matrix and associated ligands, and remodeled vigorously. See, e.g., WO 2011108993A1. Furthermore, as described (Chen et al. Adv Drug Deliv Rev. 63, 277-290 (2011)) mixed macromoleclar crowding exerts pleiotropic effects on matrix deposition. We investigated the adipogenesis-directing potential in the absence of chemical stimulus of the ECM deposited by adipogenically induced MSCs (adip) in the absence (−MMC) and presence of macromolecular crowding (+MMC). To this end, undifferentiated MSCs were seeded onto the various matrices and maintained in basal medium for 3 weeks. The cell-free matrices were produced by adipogenically induced cells in the presence/absence of crowding and undifferentiated MSCs were seeded and kept on these matrices for 7 days in basal medium. The adipocyte-derived ECM induced MSCs into spontaneous adipogenesis without chemical induction (data not shown). See, e.g., WO 2011108993A1.

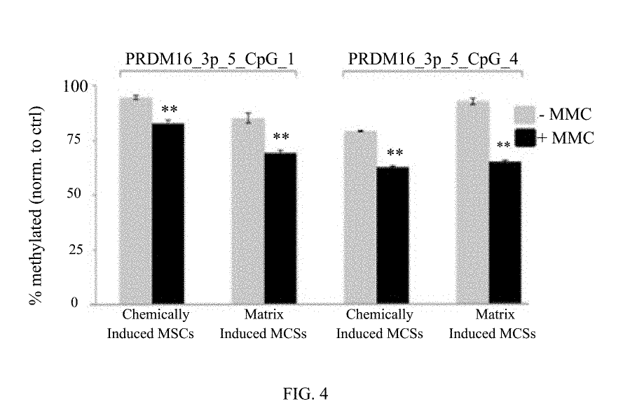

Adipocyte-derived ECM induced MSCs to express epigenetic markers of adipogenesis: We also analyzed methylation on two loci within PDRM16, a critical gene involved/upregulated during adipogenesis (Seale, P. et al. Nature. 454, 961-967 (2008). The aim of this analysis was to consider whether the epigenetic effects brought about by the classical, continued biochemical adipogenic induction would be emulated by sheer exposure of MSCs to cell-free adipocytic-derived ECMs. Interestingly, the trend of decreased methylation upon prolonged ECM contact was identical to that seen with chemical differentiation under crowding. As a decrease in methylation corresponded to an upregulation in gene expression (Cedar, H. Cell. 53, 3-4 (1988)), we inferred that the matrices deposited under crowding are indeed a strong driver of differentiation. As shown in FIG. 4, ECM was deposited by adipogenically differentiated MSCs (adip) in the absence (−) and presence (+) of macromolecular crowding (MMC); These matrices where then decellularised and fresh undifferentiated MSCs seeded on them. Sequenome analysis of CpG methylation of two loci on the gene PDRM 16, revealed a similar methylation pattern occurring in cells that had been cultured on adipocyte matrices compared to those chemically induced (n=3; error bars are ±s.d.; **P<0.01).

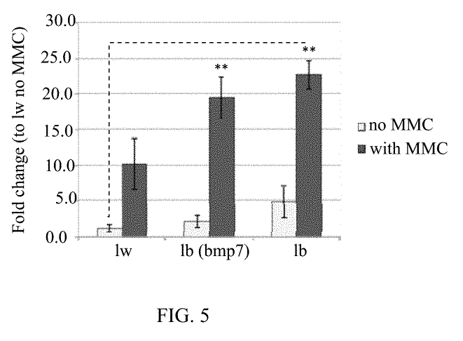

Macromolecular crowding induces the expression of UCP1 mRNA and protein, a brown adipocyte marker, in a conventional white induction protocol: Because the expression of PRDM16 was recently described in the differentiation of mouse myofibroblasts into brown adipocytes (Seale, P. et al. Nature. 454, 961-967 (2008), we analysed the expression of UCP1, a signature gene for brown adipocytes. Unexpectedly, under crowded conditions a 10-fold upregulation of UCP1 mRNA in a white differentiation protocol occurred. Switching to a brown induction protocol under crowding (see Methods, below) we observed a 23-fold upregulation in comparison to the current white induction protocols (FIG. 5).

Macromolecular crowding alone can stimulate UCP1 mRNA expression in adipogenically induced MSCs in a white and brown induction protocol, as shown in FIG. 5. Note that BMP7 (Ib group) only modestly increases UCP1 expression compared to the Ib (−BMP7) group, indicating that BMP7 may not be critical in the differentiation process. In addition, UCP1 protein was detected by immunoblotting in cell extracts of MSCs subjected to different WAT (lw) or BAT (Ib) induction protocols in the presence of mixed macromolecular crowding (+MMC) and 4 hrs of forskolin stimulation (data not shown).

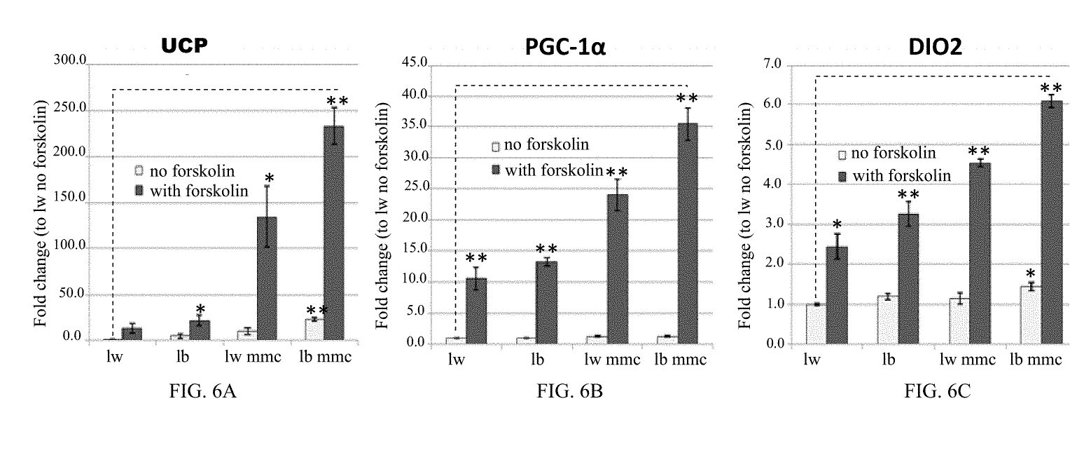

Brown adipogenic induction of MSCs under macromolecular crowding upregulates UCP1 expression several 100-fold after forskolin stimulus and other thermogenic genes. An important test for functional brown adipocytes is their response to noradrenergic stimulus or a more generic downstream induction of cAMP by forskolin stimulus (see FIG. 2). When these cells were stimulated with forskolin, a downstream signaling step following adrenergic stimulation UCP1 mRNA was upregulated an additional 10 fold. The effect of macromolecular crowding culture was striking resulting in a strong forskolin response of UCP1 upregulation after 4 hrs by two orders of magnitude. As shown in FIG. 6, macromolecular crowding under a brown induction protocol induces massive upregulation of thermogenic genes after 4 hrs of forskolin stimulation. Compared to a classical white induction protocol without MMC and forskolin stimulation (A) UCP1 mRNA expression is increased by several hundredfold (B) PGC-1α 40 times (C) DIO2 7 times (compare with FIG. 1).

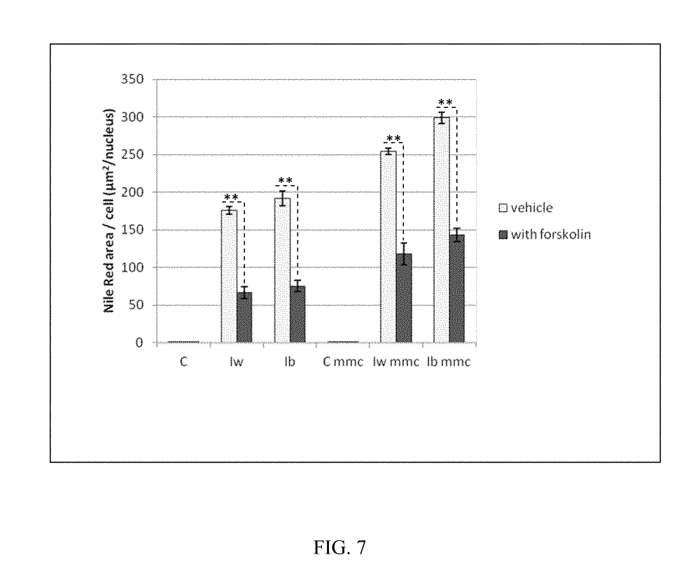

Adipocytes generated under a brown induction protocol perform lipolysis after a forskolin stimulus: We next tested whether the generic downstream induction of cAMP by forskolin stimulus (see FIG. 1) would induced lipolysis. We assessed this biochemical process by assessing the emptying of lipid stores. We quantified this by measuring a) the Nile Red areas per well (24 well) using a bioimaging station. The brown induction protocol under macromolecular crowding resulted into the hightest lipid droplet content in comparison to the current routine white induction protocol. 16 hrs after forskolin stimulus the Nile Red positive area was reduced by a factor of 2.14 in the brown induction protocol. However, these values represent a surface (2D). If we modeled this as the surface of a circle and derived a hypothetical volume of a single sphere containing triglycerides we would see a reduction of volume after forskolin treatment by a factor of 3 in the brown induced MSCs under crowding. FIG. 7 depicts forskolin treatment of white and brown adipogenically induced mesenchymal stem cells leads to emptying of lipid deposits.

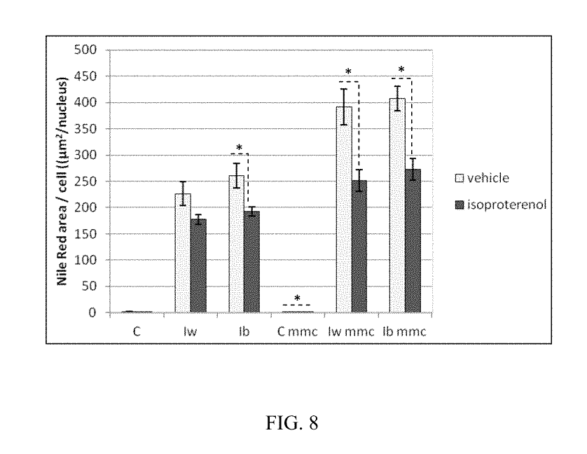

Adipocytes generated under a brown induction protocol perform lipolysis after β-adrenergic stimulus: We next tested the more BAT-specific stimulus, namely catecholamine as inducer of lipolysis. Assessment was done after 16 hrs of 5 μM isoproterenol (compare FIG. 7). Results, shown in FIG. 8, show that isoproterenol treatment of white and brown adipogenically induced mesenchymal stem cells leads to emptying of lipid droplets. Isoproterenol exerted a stronger lipolytic effect on adipocytes that had been differentiated under a BAT induction protocol.

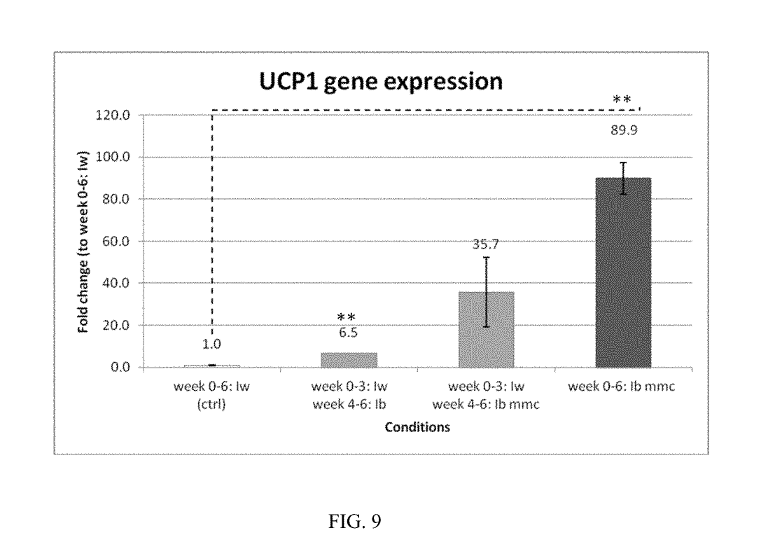

Macromolecular crowding induces a conversion of white to brown adipocytes: Lastly, to investigate whether macromolecular crowding is able to promote a white-to-brown conversion of adipocytes, MSCs were induced to differentiate for 3 weeks into white adipocytes using the standard white induction protocol (Iw), then induced for the next 3 weeks with the brown induction protocol±MMC (Ib or Ib mmc). FIG. 9 shows that exposing mature white adipocytes (at 3 weeks) to the brown induction protocol with crowding for 3 more weeks (week 0-3: Iw; week 4-6: Ib mmc) showed a 35.7-fold upregulation of UCP1 compared to just the brown induction protocol alone (week 0-3: Iw; week 4-6: Ib), which only had a 6.5-fold upregulation of UCP1.

### METHODS

a) Mesenchymal Stem Cell Culture. Human bone-marrow derived mesenchymal stem cells (MSCs) were obtained commercially (Cambrex) at passage 2 (p2) and cultured in a basic culture medium composed of low glucose Dulbecco's modified Eagle's medium (LG DMEM, Gibco) supplemented with Glutamax, 10% fetal bovine serum, 100 units/ml penicillin and 100 μl/ml streptomycin. Cells were maintained at 37° C. in a humidified atmosphere of 5% CO₂, with medium change twice per week. To prevent spontaneous differentiation, cells were maintained at subconfluent levels prior to being detached using TrypLE™ Express (Gibco), passaged at 1:3 and cultured to generate subsequent passages. Directed differentiation was carried out with cells between passage 6 (p6) and 8 (p8).

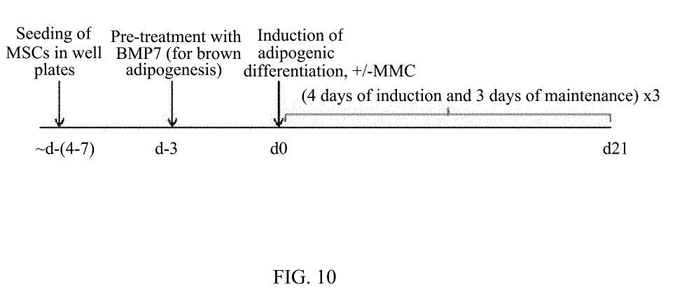

b) Adipogenic Induction of Mesenchymal Stem Cells (MSC). MSCs were seeded at an initial density of about 10.5×10⁴ cells/cm² in well plates and grown to confluence. Adipogenic differentiation was induced via three cycles of 4 days of induction, followed by 3 days of maintenance. The basal media used in the differentiation process composed of high glucose Dulbecco's modified Eagle's medium (HG DMEM, Gibco) supplemented with Glutamax, 10% fetal bovine serum, 100 units/ml penicillin and 100 μl/ml streptomycin The standard white adipogenic induction media is supplemented with 3-isobutyl-1methylxanthine (0.5 mM), indomethacin (0.2 mM), dexamethasone (1 μM) and insulin (10 μg/ml). For brown adipocyte differentiation, cells were pre-treated with BMP7 (R&D Systems 354-BP, 125 ng/ml) 3 days prior to induction. The brown adipogenic induction media is supplemented with 3-isobutyl-lmethylxanthine (0.5 mM), indomethacin (0.2 mM), dexamethasone (1 μM), insulin (10 μg/ml), triiodothyronine (1 nM) and Rosiglitazone (1 μM). Basal media alone was used during the maintenance phase. For conditions treated with macromolecular crowding (+MMC), a cocktail of macromolecules (+MMC), consisting of Ficoll 70 (37.5 mg/ml) and Ficoll 400 (25 mg/ml) was added to the basal media (High glucose DMEM, Gibco) throughout the differentiation process (during both induction and maintenance phases). A representative timeline for this strategy is shown in FIG. 10.

c) Stimulation with forskolin or isoproterenol. After the 3-week adipogenic differentiation, cells were incubated with 10 μM forskolin (Sigma) or 5 μM isoproterenol (Sigma) for 4 h or 16 h before analysis, to mimic nor-adrenergic stimulation in culture.

d) Nile Red Adherent Cytometry to assess area of cytoplasmic lipid accumulation. After 21 days (corresponding to three complete induction cycles), cell cultures were rinsed with PBS, fixed in 4% formaldehyde (10 min; RT) then co-stained for 30 min with Nile Red (Sigma-Aldrich; 5 μg/ml), for cytoplasmic lipid droplets and 4′,6-diamidino-2-phenylindole (DAPI; 0.5 μg/ml) for nuclear DNA as described previously. Adherent fluorescent cytometry was based on 9 sites per well imaged with a coolSNAP HQ camera attached to a Nikon TE2000 microscope at 2× magnification, covering 83% of total well area. Nile Red was viewed under a rhodamine filter [Ex572 nm/Em630 nm] while DAPI fluorescence was assessed with a DAPI filter [Ex350 nm/Em465 nm]. Measured Nile Red events were thresholded and measured by an image analysis software (MetaMorph 6.3v3). Extent of adipogenic differentiation was quantified by area of Nile Red fluorescence from thresholded events normalized to nuclei count based on detected DAPI fluorescence. End data corresponded to total area of lipid droplets present per well relative to cell number.

e) Quantitative PCR to assess expression of pan-adipocyte and brown adipocyte genes. Total RNA was extracted from monolayers in a 12-well plate format using Trizol (15596, Invitrogen)-chloroform method followed by the RNAeasy® mini kit (Qiagen) following the manufacturer's protocol. cDNA were synthesized from isolated mRNA using the Maxima™ First strand cDNA synthesis kit (K1642, Fermentas). Real time quantitative polymerase chain reactions (RT-PCR) were performed and monitored on a real-time PCR instrument (Stratagene) using Maxima™ SYBR Green/ROX qPCR Master Mix (K0222, Fermentas). Data analysis was carried out with the MxPro software (Strategene). Relative gene expression levels were determined using the ΔΔ-Ct method with the geometric mean of human TATA-box binding protein (TBP) and ribosomal phosphoprotein P0 (RPLP0) levels as an endogenous control. Primer sequences used are shown in Table 1.

TABLE 1
Primer sequences (written 5′→3′, Forward and Reverse)

| Gene | Accession no. | Sequence | Reference |
|---|---|---|---|
| RPLP0 | NM_001002.3 | CAC CAT TGA AAT CCT GAG TGA TGT | Jansen et al., 20 [symbol] |
| TBP | NM_003194.4 | CAC GAA CCA CGG CAC TGA TT | Elabd et al., 20 [symbol] |
| | | TTT TCT TGC TGC CAG TCT GGA C | |
| UCP1 | NM_021833.4 | CTG GAA TAG CGG CGT GCT T | Virtanen et. al. [symbol] |
| | | AAT AAC ACT GGA CGT CGG GC | |
| PGC-1α | NM_013261.3 | GCC AAA CCA ACA ACT TTA TCT CTT C | Virtanen et. al. [symbol] |
| | | CAC ACT TAA GGT GCG TTC AAT AGT C | |
| DIO2 | NM_013989.4 | CCT CCT CGA TGC CTA CAA AC | Virtanen et. al. [symbol] |
| | | GCT GGC AAA GTC AAG AAG GT | |

RPLP0 (human ribosomal phosphoprotein P0); TBP (TATA-box binding protein);
UCP1 (uncoupling protein 1); PGC-1α (PPAR-γ co-activator 1α); DIO2
(Deiodinase, iodothyronine, type II) [symbol]
indicates data missing or illegible when filed

f) Protein extraction and Western blotting. Protein was extracted as whole cell lysates from cell monolayers using Laemmli buffer. 17.6 μl of protein extract for each sample was subjected to a reducing SDS-PAGE. Proteins were then transferred onto a nitrocellulose membrane (Bio-Rad) for 16 h at 20V. Membranes were blocked with 5% non-fat milk in TBST for 1 h at RT. The membrane was then incubated with the primary antibody in 1% non-fat milk in TBST for 1.5 h at RT. Primary antibodies used were anti-rabbit UCP1 (1:100, ab10983 Abcam), anti-mouse COXIV (1:1000, ab33985 Abcam), and anti-mouse β-actin (1:1000 A228Sigma) as a loading control. Bound primary antibody was detected with Dako HRP goat-anti mouse antibody (P0447) and Dako HRP goat-anti rabbit antibody (P0448) at 1:1000 in 1% non-fat milk in TBST for 1 h at RT. Chemiluminescence was captured with a Versadoc (Biorad 5000MP).

### REFERENCES

1. Cypess, A. M. & Kahn, C. R. Brown fat as a therapy for obesity and diabetes. Current Opinion in Endocrinology, Diabetes and Obesity. 17, 143-149 (2010).

2. Lareu, R. R. et al. Emulating a crowded intracellular environment in vitro dramatically improves RT-PCR performance. Biochem Biophys Res Commun. 363, 171-177 (2007).

3. Harve, K. S. et al. Understanding how the crowded interior of cells stabilizes DNA/DNA and DNA/RNA hybrids-in silico predictions and in vitro evidence. Nucleic Acids Res. 38, 172-181 (2010).

4. Lareu, R. R. et al. In vitro enhancement of collagen matrix formation and crosslinking for applications in tissue engineering: a preliminary study. Tissue Eng. 13, 385-391 (2007).

5. Lareu, R. R. et al. Collagen matrix deposition is dramatically enhanced in vitro when crowded with charged macromolecules: the biological relevance of the excluded volume effect. FEBS Lett. 581, 2709-2714 (2007).

6. WHO Factsheet: Obesity and Overweight. http://www.who.int/mediacentre/factsheets/fs311/en/index.html#(2011)

7. Rothwell, N. J. & Stock, M. J. Luxuskonsumption, diet-induced thermogenesis and brown fat: the case in favour. Clin Sci (Lond). 64, 19-23 (1983).

8. Celi, F. S. Brown adipose tissue—when it pays to be inefficient. N Engl J Med. 360, 1553-1556 (2009).

9. Gesta, S., Tseng, Y. H. & Kahn, C. R. Developmental origin of fat: tracking obesity to its source. Cell. 131, 242-256 (2007).

10. Nedergaard, J., Bengtsson, T. & Cannon, B. Unexpected evidence for active brown adipose tissue in adult humans. AJP: Endocrinology and Metabolism. 293, E444-E452 (2007).

11. Saito, M. et al. High incidence of metabolically active brown adipose tissue in healthy adult humans: effects of cold exposure and adiposity. Diabetes. 58, 1526-1531 (2009).

12. Zingaretti, M. C. et al. The presence of UCP1 demonstrates that metabolically active adipose tissue in the neck of adult humans truly represents brown adipose tissue. FASEB J. 23, 3113-3120 (2009).

13. van Marken Lichtenbelt, W. et al. Cold-activated brown adipose tissue in healthy men. N Engl J Med. 360, 1500-1508 (2009).

14. Cypess, A. et al. Identification and importance of brown adipose tissue in adult humans. N Engl J Med. 360, 1509-1517 (2009).

15. Virtanen, K. A. et al. Functional brown adipose tissue in healthy adults. N Engl J Med. 360, 1518-1525 (2009).

16. Yki-Järvinen, H. Thiazolidinediones. N Engl J Med. 351, 1106-1118 (2004).

17. Bogacka, I., Xie, H., Bray, G. A. & Smith, S. R. Pioglitazone induces mitochondrial biogenesis in human subcutaneous adipose tissue in vivo. Diabetes. 54, 1392-1399 (2005).

18. Vernochet, C. et al. C/EBPalpha and the corepressors CtBP1 and CtBP2 regulate repression of select visceral white adipose genes during induction of the brown phenotype in white adipocytes by peroxisome proliferator-activated receptor gamma agonists. Mol Cell Biol. 29, 4714-4728 (2009).

19. Petrovic, N. et al. Chronic peroxisome proliferator-activated receptor gamma (PPARgamma) activation of epididymally derived white adipocyte cultures reveals a population of thermogenically competent, UCP1-containing adipocytes molecularly distinct from classic brown adipocytes. J Biol Chem. 285, 7153-7164 (2010).

20. Chen, C., Loe, F., Blocki, A., Peng, Y. & Raghunath, M. Applying macromolecular crowding to enhance extracellular matrix deposition and its remodeling in vitro for tissue engineering and cell-based therapies. Adv Drug Deliv Rev. 63, 277-290 (2011).

21. Seale, P. et al. PRDM16 controls a brown fat/skeletal muscle switch. Nature. 454, 961-967 (2008).

22. Cedar, H. DNA methylation and gene activity. Cell. 53, 3-4 (1988).

23. Bonet, M.L. et al., Pharmacological and nutritional agents promoting browning of white adipose tissue, Biochim. Biophys. Acta (December 2012)

24. Jansen, P. A. M. et al. Expression of the vanin gene family in normal and inflamed human skin: induction by proinflammatory cytokines, Journal of Investigative Dermatology (2009) 129, 2167-2174.

25. Elabd, C. et al., Oxytocin controls differentiation of human mesenchymal stem cells and reverses osteoporosis, Stem Cells 2008;26:2399-2407.

26. Loe et al PCT/SG2011/000081; WO20111/108993 A1

27. US 2010/0150885 A1

28. Raghunath, M et al. WO 2008/018839 A1

1. A method of generating functional human brown adipocytes from bone marrow-derived mesenchymal stem cells, comprising culturing the cells with a differentiation cocktail comprising one or more adipogenic agents and one or more browning agents selected from one or more macromolecular crowders at a total concentration of about 10-80 mg/ml, wherein the cells thereby differentiate into functional human brown adipocytes.

2.-4. (canceled)

5. The method of claim 1, wherein the one or more adipogenic agent(s) is selected from the group consisting of: insulin, glucocorticoid or synthetic equivalent, a cAMP enhancer, and vitamin C.

6. (canceled)

7. The method of claim 1, wherein the differentiation cocktail further comprises one or more additional browning agent(s) selected from thyroid hormone, a PPARγ receptor agonist, a bone morphogenetic protein, a retinoid, a cardiac natriuretic peptide, a myokine, a fibroblast growth factor, a microRNA, a lactogen, an insulin-like growth factor, orexin, a bile acid, nitric oxide, a hyperacetylating agent, a hypomethylating agent, a prostaglandin, a PPARα ligand, TLQP-21, brain-derived neurotrophic factor, leptin, a β-adrenergic agonist, an AMPK activator, capsaicin or an analog thereof, fucoxanthin, 2-hydroxyoleic acid, resveratrol, conjugated linoleic acid, an n-3 fatty acid of marine origin, scallop shell powder and bofutsushosan.

8. The method of claim 7, wherein the browning agent comprises a PPARγ receptor agonist that is a thiazolidinedione selected from rosiglitazone, ciglitazone, pioglitazone, darglitazone or troglitazone.

9. The method of claim 1, wherein the macromolecular crowders comprise:
(a) an organic-based hydrophilic macromolecule having a molecular weight of 50 kDa to 500 kDa and a neutral surface charge;
(b) an organic-based hydrophilic macromolecule having a radius range of 2 to 50 nm and a neutral or negative surface charge; or
(c) a mixture of two or more of organic-based hydrophilic macromolecules described in (a) and/or (b).

10. The method of claim 9, wherein one or more of the organic-based hydrophilic macromolecule(s) is:
a) a carbohydrate-based hydrophilic macromolecule;
b) a polymer of glucose and/or sucrose; or
c) neutral or derivatised glucans; fructans; levans; glycosaminoglycans; or mixtures thereof.

11.-13. (canceled)

14. The method of claim 1, further comprising verifying the functionality of the human brown adipocytes by a method comprising:
a) stimulating the human brown adipocytes with a specific β-adrenergic receptor agonist and/or compound which elevates intracellular levels of cAMP, and
b) quantifying one or more of an activity selected from the group consisting of: the expression of the UCP1 gene/protein; mitochondrial biogenesis; oxygen consumption; uncoupled respiration; glucose uptake; lipolysis; and fuel metabolism of the human brown adipocytes,
wherein the functionality of the human brown adipocytes is verified when: the expression of the UCP1 gene/protein; mitochondrial biogenesis; oxygen consumption; uncoupled respiration; glucose uptake; lipolysis; or fuel metabolism of the human brown adipocytes is increased compared with a comparable measurement obtained in the absence of simulation by the specific β-adrenergic receptor agonist and/or compound which elevates intracellular levels of cAMP.

15. The method according to claim 14, wherein a specific β-adrenergic receptor agonist is used, and is selected from isoprenaline, noradrenalin, adrenalin, dobutamine, terbutaline, compound CL316243, or isoproterenol.

16. The method according to claim 15, wherein a compound which elevates intracellular levels of cAMP is used, and is selected from dibutyryl-cAMP, 8-CPT-cAMP, 8-bromo-cAMP, dioctanoyl-cAMP, indomethacin, IBMX, or forskolin.

17.-38. (canceled)

39. A method for screening for agents capable of altering the metabolic activity of an individual, comprising activating the thermogenic programme of a population of functional brown adipocytes prepared by a method comprising culturing bone marrow-derived mesenchymal stem cells with a differentiation cocktail comprising one or more adipogenic agents and one or more browning agents selected from one or more macromolecular crowders at a total concentration of about 10-80 mg/ml, wherein the cells thereby differentiate into functional human brown adipocytes.

40. A method of autologous cell-based therapy for the clinical treatment of a metabolic disease in an individual in need thereof, comprising introducing a population of functional brown adipocytes into the individual, wherein the adipocytes are prepared by a method comprising culturing bone marrow-derived mesenchymal stem cells with a differentiation cocktail comprising one or more adipogenic agents and one or more browning agents selected from one or more macromolecular crowders at a total concentration of about 10-80 mg/ml, wherein the cells thereby differentiate into functional human brown adipocytes.

41. The method of claim 1, wherein the differentiation cocktail is incorporated into a pharmaceutical composition, wherein the pharmaceutical composition comprises a biomaterial permeated or impregnanted with the differentiation cocktail.

42. The method of claim 41, wherein the biomaterial is selected from a hydrogel, an electrospun mesh, nanoparticles or microparticles.
